(19) United States
(12) Patent Application Publication
(10) Pub. No.: US 2024/0374850 A1
(43) Pub. Date: Nov. 14, 2024

(54) Retrofittable Suction Catheter

(71) Applicant: Jeffrey Carl Cowgill, Vancouver, WA (US)

(72) Inventor: Jeffrey Carl Cowgill, Vancouver, WA (US)

(21) Appl. No.: 18/338,585

(22) Filed: Jun. 21, 2023

Related U.S. Application Data
(63) Continuation-in-part of application No. 18/301,933, filed on Apr. 17, 2023.

(51) Int. Cl. A61M 16/04; A61M 1/00

(52) U.S. Cl. CPC A61M 16/0463; A61M 1/742; A61M 1/87; A61M 16/049

(57) ABSTRACT

A patient operated, portable, phlegm elimination device that may be attached in various ways to the body of any manufacturer's respiratory device such as an aerosol delivery unit or a positive exhalation pressure (PEP) device. It allows the patient to clear their airways of phlegm during respiratory treatments or exercises without the use of a separate device or releasing the respiratory device currently in their hands. It is simple to operate and is clear for purposes of evaluating the operability, cleanliness and prior usage of the

### PRIORITY CLAIM TO A NONPROVISIONAL APPLICATION

THIS APPLICATION IS A CONTINUATION IN PART OF U.S. patent application Ser. No. 18/301,933, ENTITLED DUAL PURPOSE RESPIRATORY TREATMENT DEVICE, FILED Apr. 17, 2023, WHICH IS INCORPORATED IN ITS ENTIRETY HEREIN BY REFERENCE.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to the medical field, and more particularly to respiratory technology.

### BACKGROUND

Respiratory treatments vary considerably as do the different manufacturer's devices used to facilitate the treatments. These treatments accomplish one or a combination of three goals. First, they may help to strengthen the patient's lungs. Second, they may deliver a respiratory aerosol medicament into the patient's lungs. Third, they may clear the phlegm and mucus from the patient's airways. These are commonly accomplished with the patient undergoing separate regimens of portable, phlegm elimination systems, possibly with a subsequent respiratory medicament inhalation (drug delivery). The PEP treatment requires the patient to exhale through any of a plethora of PEP devices that send a resultant pulsation pressure wave back down their lungs. If this pressure wave has sufficient power, it will dislodge the phlegm from their airways. This phlegm must be eliminated. When enough cycles have been performed, the patient's breathing ability is improved as their lungs are cleared of phlegm to the point where they can undergo an inhalation treatment and receive a medicated aerosol (generally inhaled antibiotics, bronchodilators, corticosteroids) to further increase their lung capacity and ease their labored breathing.

There are several problems encountered by patients using such PEP devices. When the phlegm is brought out of the patient's airways it must exit the mouth and needs a place to be expelled. There is not always a handy place nearby to receive the phlegm. Patients are often elderly, weak or partially incapacitated. Because of their diminished lung capacity, successful exhalations (events) often leaves them out of breath and gasping for air. At this time, dealing with a mouth full of phlegm, while holding a PEP device and trying to catch one's breath is daunting for many. This also stops the rhythm of the PEP routine which is important as the effect of each exhalation builds on itself. A repeated pattern of inhalation and exhalation is necessary to gain the full medical effect of the PEP device. This should not be interrupted.

If the PEP device or the medicament delivery device is contaminated with mucus they may cease to function or at the least, require intensive cleaning subject to prevent bacterial growth. There are vacuum units to deal with the phlegm but switching between these devices and a vacuum unit increases the treatment complexity and robs precious time between breaths.

For these reasons, self-treatment with a PEP device or an aerosol medicament device by a severely respiratory compromised individual, is not a possibility, or if attempted will not provide the full beneficial effects.

Thus, a novel device that allows the full spectrum of respiratory training and drug delivery devices to be operated by the patient themselves, would fulfill a long felt need in the medical industry. This new invention utilizes and combines known and new technologies in a unique and novel configuration to overcome the aforementioned problems and accomplish this.

### BRIEF SUMMARY

A suction catheter system that may be integrated onto the exterior of the body of a PEP device or Nebulizer for patients with severe airway clearance issues, that protects against potential airway aspiration and can be used for in hospital use and home care.

In accordance with various embodiments, a suction catheter system is provided that is attachable to any style of respiratory device, and utilizes a separate pathway external to that device's respiratory pathway to eliminate the phlegm developed.

In one aspect, a self-administered respiratory treatment device that allows a patient to maintain the regular, rhythmed breathing to maximize the benefit of the PEP technology or aerosol drug administration technology, despite the occurrence of respiratory phlegm.

In another aspect, a self-operable suction catheter, retrofittable onto the exterior face of the body of any portable respiratory device, is provided.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a side perspective view of the first embodiment retrofittable suction catheter device;

FIG. 2 is a front view of the first embodiment respiratory treatment device;

FIG. 3 is a side view of a vacuum unit;

FIG. 4 is a side view of a first embodiment respiratory treatment device in use;

FIG. 5 is a side perspective view of a second embodiment respiratory treatment device;

FIG. 6 is a side view of a second embodiment respiratory treatment device in use;

FIG. 7 is a side view of the preferred embodiment catheter retention strip;

FIG. 8 is a side view of the alternate embodiment catheter retention strip;

FIG. 9 is an end view of a retention strip with a catheter sleeve attached atop;

FIG. 10 is an alternate embodiment catheter strip atop a substrate strip with a male press fitting thereon;

FIG. 11 is an alternate embodiment catheter sleeve atop a substrate strip with a screw mount; and

FIG. 12 is an alternate embodiment catheter sleeve with an attached hook and loop fastener.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The accompanying drawings are not necessarily drawn to scale. The examples provided are for illustrative purposes and are not intended to limit the scope of the invention.

It will be understood that when an element or layer is referred to as being “on,” “coupled to,” or “connected to” another element or layer, it can be directly on, directly coupled to or directly connected to the other element or layer, or intervening elements or layers may be present. In contrast, when an element is referred to as being “directly on,” “directly coupled to,” or “directly connected to” another element or layer, there are no intervening elements or layers present. Like numbers refer to like elements throughout. As used herein, the term “and/or” includes any and all combinations of one or more of the associated listed items.

As used herein, the term “PEP or OPEP device” refers to a class of respiratory illness treatment devices that requires the patient to exhale through them so as to cause a resultant pulsation pressure wave back down their lungs for the purpose of dislodging the phlegm from their airways to be coughed up.

As used herein, the term “Nebulizer” refers to any of a class of respiratory aerosol drug delivery devices that may be operated as a stand-alone device or incorporated with another respiratory unit such as a PEP or OPEP device.

As used herein, the terms “release strip and adhesive release strip” refers to a linear strip (generally polymer) that is removably placed atop an adhesive strip. When removed, it exposes the adhesive to be able to adhere to a object it comes in contact with.

As used herein, the term “open sleeve” refers to a sleeve with two open ends.

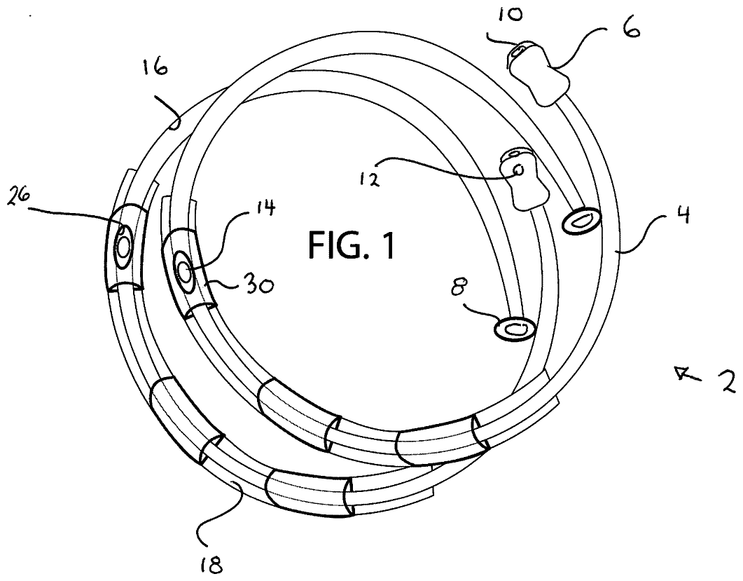

The present invention relates to a novel design for a suction catheter device that can be retrofit onto the side of the body of any manufacturer's PEP, OPEP or Nebulizer. Doing this blends together complimentary devices used in the treatment of respiratory diseases. Looking at FIG. 1, a pair of suction catheter devices 2 can be seen alone in a coiled (packaged) configuration. It consists of a length of clear, serializable, polymer tube 4 that has a suction mouthpiece 6 affixed at the proximal (patient) end, and a vacuum tip, 8 removably connected at the distal (vacuum system) end that is configured for mating connection with a medical vacuum system's inlet connector fittings. Between the distal end and the proximal end there is a single suction control port 14 formed through the side wall 16 of the suction catheter's polymer tubing. In normal operation, when connected to a vacuum system, covering this suction control port 14 is the sole method the suction catheter device has to initiate suction through the suction tip 6 and draw phlegm from the patient's mouth. Also, between the distal and proximal ends there is a catheter retention strip 18 with a respiratory device attachment means on a bottom face thereof and having a series of at least two and preferably three clear catheter open sleeves 20 and 22 mounted thereon. There is at least one and preferably, two sleeves 20 and one catheter control sleeve 22. In the preferred embodiment all these sleeves 20 and 22 have a cross sectional geometric configuration that will accommodate the suction catheter 4 therein. In the preferred embodiment this is semi-circular or expressed differently, it has dome shaped top face and a planar bottom face 30 (FIG. 1) The catheter control sleeve 22 has a cutout 26 at the apex of its domed top face 28 that overlays, and is larger than the suction control port 14. All the catheter sleeves 20 and 22 have their bottom faces 30 affixed to the planar top face 24 of the catheter retention strip 18.

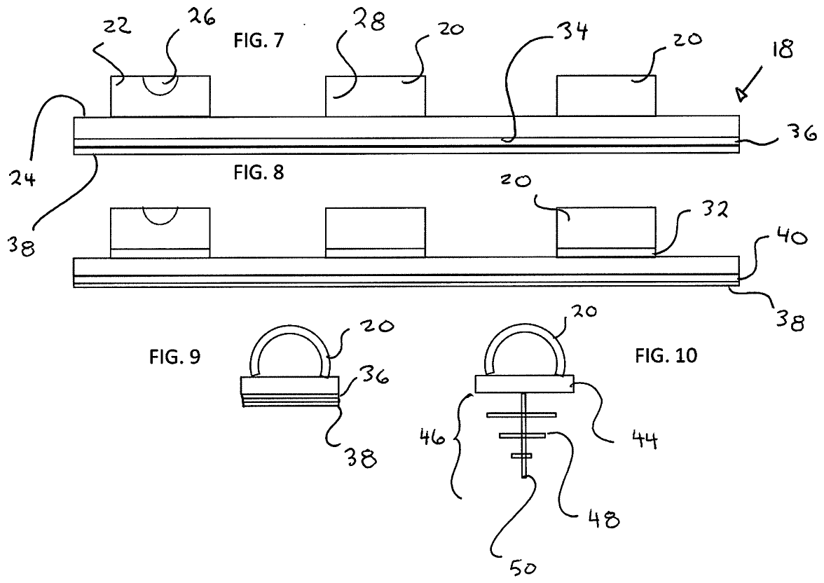

The catheter retention strip 18 is best seen in FIGS. 7-9. It is a flexible, polymer strip having a planar top face 24 with three fixed tube catheter sleeves 20 and 22 affixed thereon at spaced intervals. In the preferred embodiment of FIG. 8 the catheter sleeves 20 and 22 can be seen permanently affixed to the top face of the retention strip 18. The spacing between them is a fixed distance. In alternate embodiments, the catheter sleeves 20 and 22 have an adhesive 32 between their bottom face 30 and the top face 24 of the retention strip 18 to allow for strategic placement of them on the bodies of odd shaped respiratory devices. It also allows for the use of more than two catheter sleeves 20 to be used where the catheter retention strip 18 is longer or has a serpentine path.

The retention strip 18 in the preferred embodiment (FIG. 7) has as its respiratory device attachment means, a strip of double sided tape 36 stuck to its bottom face 34. The outer face of the double sided tape 36 has a release strip 38 on its adhesive bottom face. This is removed when the device 2 is to be mounted on a respiratory device. In alternate embodiments, (FIG. 8) a liquid adhesive 40 is applied by rolling or spraying onto the bottom face 34 of the retention strip 18 and a release strip 38 is placed onto the adhesive 40. FIG. 9 shows an end view of the retention tape strip 18 and a catheter sleeve 20 using double sided adhesive tape 36. However, it is known that there are a plethora of other respiratory device attachment means that may be used and incorporated onto the bottom face of the retention strip 18, including but not limited to the male press fittings (discussed herein), hook and loop fastener strips (discussed herein) and mechanical fasteners such as screws, rivets and the like. (Not illustrated on the retention strip 18.)

The suction tip 6 has an hourglass configuration that narrows about its center. This is designed to help maintain the suction tip 6 in contact with the patient's lips as the vacuum draws phlegm from their mouth and into the suction catheter 2. There is a suction orifice 10 formed through the body of the suction tip. In the preferred embodiment this suction orifice 10 lies along the linear axis of the suction tip, although in other embodiments there may be a transverse, angled or combination orifice 12 used.

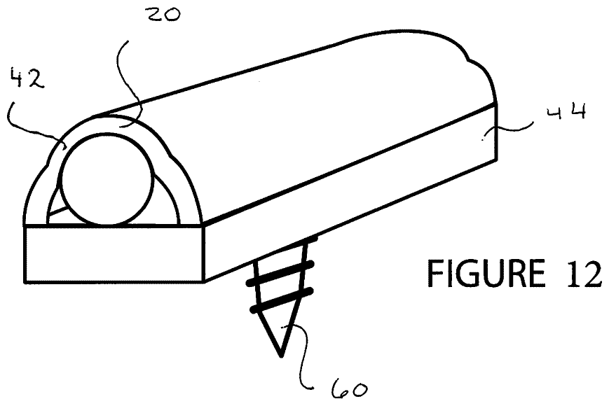

While all the catheter sleeves 20 and 22 are dome shaped so as to frictionally engage the top of the suction catheter 4, the true semi-circular cross sectional dome configuration as seen in FIG. 9 may be modified as seen in FIG. 12 to have a smaller diameter arc 42 in its top half. This serves to increase the area of contact between the inner face of the catheter sleeve 20 and the outer wall of the suction catheter 4.

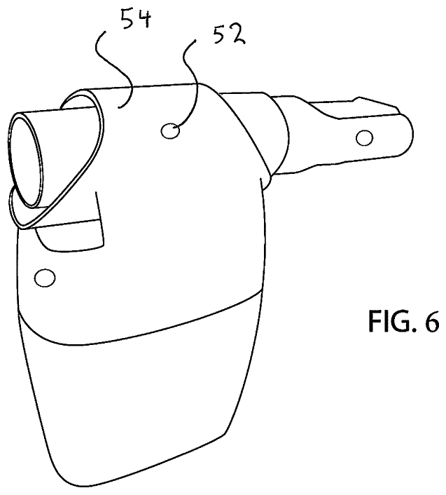

There are three other alternate embodiment catheter sleeve assemblies (FIGS. 10-12) used when the retention strip 18 is to be eliminated. The first alternate embodiment catheter sleeve 20 is affixed atop a rigid or semi-rigid substrate strip 44 that has a male press fitting 46 formed on the bottom. These press fittings 46 are also called automotive body panel fittings and typically have at least three increasingly larger diameter disks 48 extending radially outward from a peg 50 that extends perpendicularly from the substrate strip 44. These would be used on a PEP device 54 that has been prepared by the drilling of mounting orifices 52 through its body as illustrated in FIG. 6. Since the press fitting portion 46 is inserted into the mounting orifice until its disks 48 can no longer fit through, there is minimal air leakage at this connection point.

The second alternate embodiment catheter sleeve 20 is affixed atop a rigid or semi-rigid substrate strip 44 that has a screw 60 formed on or through the bottom face thereof. This type of catheter sleeve is also used with a respiratory device with mounting orifices drilled therethrough one side of its body as seen in FIG. 6.

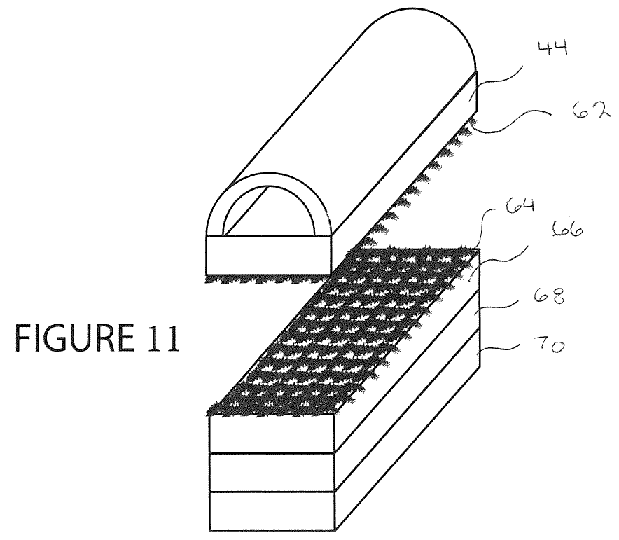

The third alternate embodiment catheter sleeve 20 (FIG. 11) has the catheter sleeve 20 affixed atop a rigid or semi-rigid first substrate strip 44 that has a first portion of a hook and loop fastener 62 formed on or attached to the bottom face thereof. There is a second portion of a hook and loop fastener 64 formed or attached to the top face of a rigid or semi-rigid second substrate strip 66 that has an adhesive 68 and release strip 70 thereon. This second portion of a hook and loop fastener 64 has its release strip removed and it is stuck to the body of the respiratory device wherever the suction catheter 4 is to be mounted.

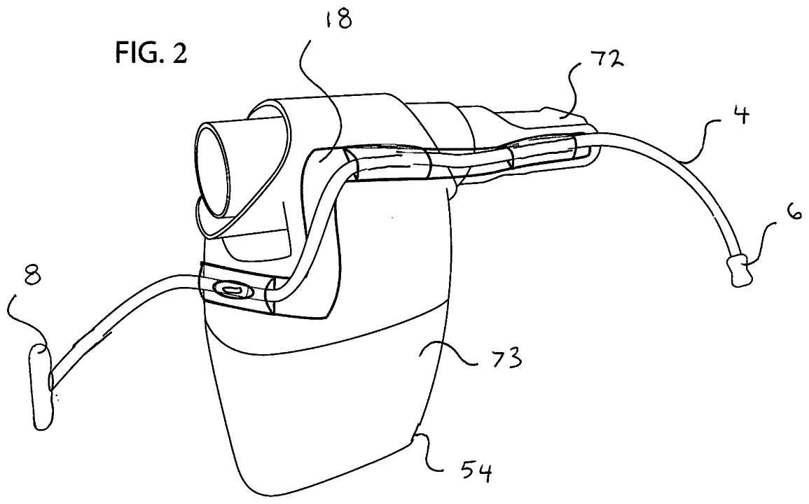

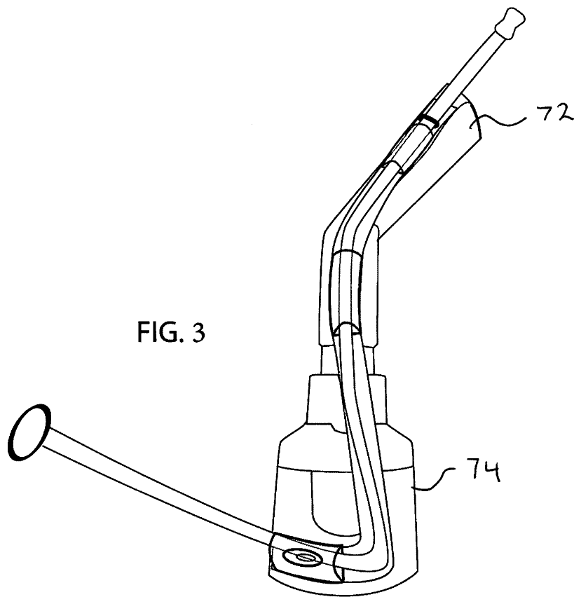

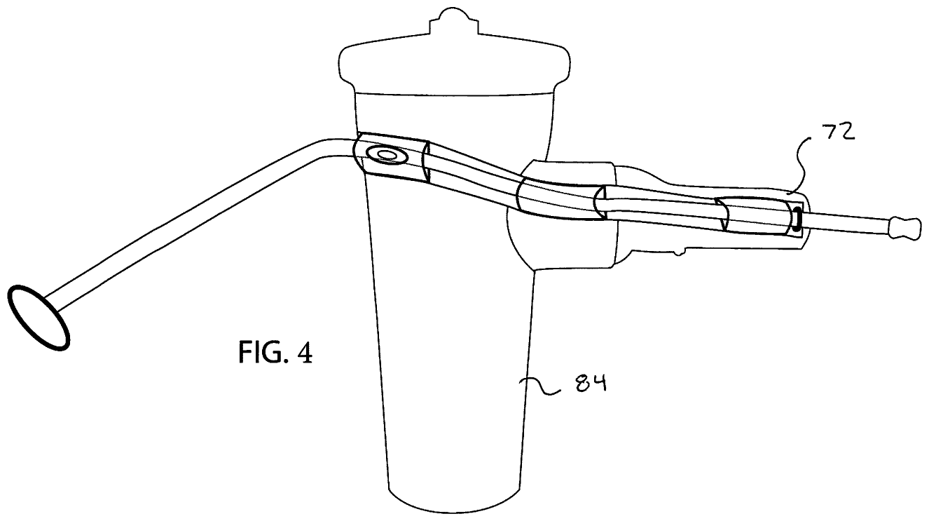

FIGS. 2-4 illustrate the preferred embodiment suction catheter device 2 affixed to a PEP device (FIG. 2) and affixed to two different configurations of nebulizers (FIGS. 3 and 4). To install the suction catheter device 2, the release strip 38 on the bottom of the retention strip 18 is peeled off and the strip is conformed to the side wall of the respiratory device 54, 74 or 84 such that the catheter control sleeve 30 and its cutout 26 with the suction control port 14 centered therein, is placed at a location where the patient would normally grasp the respiratory device and rest their index finger. The remainder of the retention strip 18 is shaped around the surface of the respiratory device such that the proximal end with the suction tip 6 is in close proximity to the patient's mouthpiece 72.

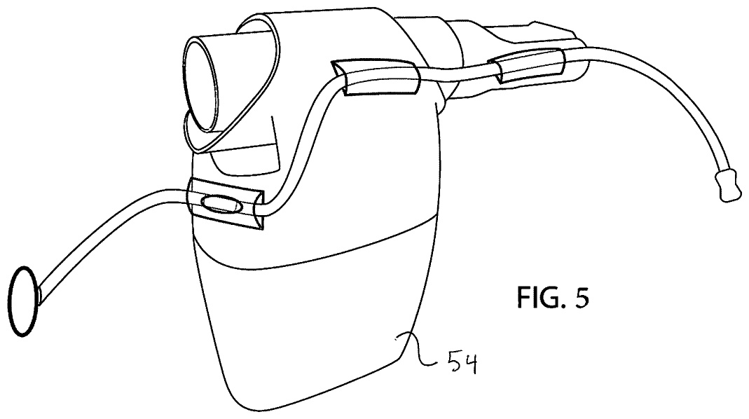

FIG. 5 illustrates a PEP device 54 with an alternate embodiment suction catheter device affixed thereto. In this embodiment there is no retention strip 18 and any of the three alternate embodiment catheter sleeves, may be affixed to the body of the respiratory device with adhesive, push fillings or mechanical fasteners such as screws.

The catheter sleeves 20 and 22 and the suction catheter 4 are clear such that the volume, color and consistency of the phlegm can be seen and noted. It also shows if the device is plugged, dirty or has been used.

With any embodiment of the suction catheter device 2 mounted on a respiratory device, a dual purpose respiratory device will have been made. This has not been seen in the medical field. In use, the suction catheter device 2 is affixed properly to a respiratory device and the distal end of the suction catheter is connected to an operating vacuum system via its vacuum tip 8. (not illustrated) The patient uses the PEP or nebulizer until they sense a buildup of loose phlegm in their lings. The respiratory device's mouthpiece is removed from the patient's mouth, and the patient inserts the suction tip mouthpiece 6 into their mouth, and close their lips as they cough up phlegm. They direct the phlegm in their mouth to a location near the suction mouthpiece 6 so it can be quickly drawn away to the suction tank. At the same time, they put their finger over the suction control port 14 and block it commensurate with the amount of vacuum they want felt in their mouth to draw away the phlegm. In this way the control orifice provides variable suction control.

It is to be understood that depending on which style of airway clearance device is used, the physical configuration of the polymer body will differ, however, all these devices share commonalities that allow them to be modified consistent with the suction catheter device's design provided herein to accomplish the same end result.

While certain features and aspects have been described with respect to exemplary embodiments, one skilled in the art will recognize that numerous modifications are possible. As such, this detailed description and accompanying material is intended to be illustrative only, and should not be taken as limiting the scope of the inventive concept. What is claimed as the invention, therefore, is all such modifications as may come within the scope and spirit of the following claims and equivalents thereto.

1. A suction catheter assembly for retrofitting a respiratory device into a dual purpose respiratory device, comprising:
a flexible suction catheter tube having a distal end and a proximal end;
a suction control port formed through said suction catheter tube;
a flexible suction catheter retention strip having a top planar face and a bottom planar face with a respiratory device attachment means thereon;
at least one catheter open sleeve affixed thereon said top planar face of said catheter retention strip;
a catheter control open sleeve with a control cutout therein, said catheter control open sleeve affixed thereon said top planar face of said catheter retention strip;
a suction mouthpiece affixed at a proximal end of said suction catheter tube;
a vacuum tip, affixed at said distal end of said suction catheter tube; and
wherein said suction catheter tube resides atop said suction catheter retention strip and passes through said catheter control open sleeve and through said at least one catheter open sleeve.

2. The suction catheter assembly of claim 1 wherein said catheter suction tube, said catheter open sleeve and said catheter control open sleeve are clear.

3. The suction catheter assembly of claim 2 wherein said catheter control open sleeve and said at least one catheter open sleeve are dome shaped; and
wherein said control cutout is located at an apex of said dome shape of said catheter control open sleeve.

4. The suction catheter assembly of claim 1 wherein said respiratory device attachment means is an adhesive with an adhesive release strip thereon.

5. The suction catheter assembly of claim 1 wherein said respiratory device attachment means is a hook and loop fastener.

6. The suction catheter assembly of claim 1 wherein said respiratory device attachment means is a mechanical fastener.

7. A suction catheter assembly for retrofitting a respiratory device into a dual purpose respiratory device, comprising:
a flexible suction catheter tube having a distal end and a proximal end;
a suction control port formed through said suction catheter tube;
at least one catheter open sleeve with a respiratory device attachment means thereon;
a catheter control open sleeve with a control cutout therein and said respiratory attachment means thereon;
a suction mouthpiece affixed at a proximal end of said suction catheter tube;
a vacuum tip, affixed at said distal end of said suction catheter tube; and
wherein said suction catheter tube passes through said catheter control open sleeve and through said at least one catheter open sleeve; and
wherein said suction control port resides within said catheter control open sleeve and aligned with said control cutout.

8. The suction catheter assembly of claim 7 wherein said respiratory device attachment means is selected from the group consisting of hook and loop fastener strips, mechanical fasteners, screws, rivets, double sided tape and adhesives.
